(19) United States
(12) Patent Application Publication
(10) Pub. No.: US 2003/0191076 A1
(43) Pub. Date: Oct. 9, 2003

(54) Prime-boost vaccination strategy

(76) Inventors: Steve Wesselingh, Vic (AU); Ian Barry Dry, S.A. (AU); Richard Anthony Strugnell, Vic (AU); Ian Allister Ramshaw, Act (AU)

Correspondence Address: Bingham McCutchen, Suite 1800, Three Embarcadero Center, San Francisco, CA, 94111-4067, US

(21) Appl. No.: 10/181,633

(22) PCT Filed: Jan. 22, 2001
(86) PCT No.: PCT/AU01/00059

(30) Foreign Application Priority Data
Jan. 21, 2000 (AU) PQ 5208

(51) Int. Cl. A61K 48/00; A01H 1/00; C12N 15/82; A61K 35/78

(52) U.S. Cl. 514/44; 800/288; 424/750; 424/751; 424/777; 424/774

(57) ABSTRACT

The present invention provides a method for inducing an immune response to an antigen in a subject. The method comprises administering to the subject DNA encoding the antigen, and subsequently orally administering to the subject a composition comprising transgenic material, wherein the transgenic material comprises a DNA molecule encoding the antigen such that the antigen is expressed in the transgenic material.

### FIELD OF THE INVENTION

The present invention relates to a method for inducing an immune response to an antigen in a subject.

### BACKGROUND OF THE INVENTION

Measles is a highly contagious viral disease that has persisted for more than 1000 years since it was first described (Babbott and Gordon, 1954). Severe infection may lead to pneumonia, encephalitis (brain inflammation) and death. Although measles can be effectively prevented by a live-attenuated vaccine (LAV) it still causes approximately 800,000 deaths every year, predominantly among children in developing countries (Cutts and Steinglass, 1998).

The inability to control measles using the LAV is largely due to neutralization of the vaccine by maternal antibodies. In order to avoid neutralization by maternal antibodies the LAV is generally administered between 12 and 18 months, However maternal antibodies may decline more rapidly in infants of developing countries (Gans et al., 1998). As a consequence, there is a window between 6 and 18 months of age during which infants may lack both passive and active immunity.

An additional concern is the effective distribution and use of live attenuated measles vaccines in developing countries in particular the maintenance of the “cold chain” during transport and storage to ensure the viability of the vaccine prior to administration. This, together with requirement for trained staff for parenteral application of the vaccine, has led to poor vaccination coverage in these countries.

In an attempt to overcome the problem of maternal antibodies a high titre Edmonston-Zagreb vaccine was given to young infants in the late 1980's. This vaccine protected infants against measles but led to an increased mortality from other infections such as diarrhoea and pneumonia (Markowitz et al., 1990; Garenne et al., 1991) and was subsequently withdrawn from use in 1992 (Weiss, 1992). It is thought that the increase in mortality was due to an immunosuppressive effect similar to that seen with wild type infection.

Sub-unit vaccines are not subject to the same constraints as LAVs. Development of a sub-unit vaccine for measles would primarily address issues concerning the immunization and protection of children in the developing world, such as maternal antibodies. In addition to this non-replicating sub-unit vaccines cannot initiate infection in immuno-compromised patients. New vaccine approaches such as DNA subunit vaccines and edible subunit vaccines are currently being devised as alternatives to the LAV. The measles virus (Mv) hemagglutinin (H) protein is an immunodominant surface exposed glycoprotein and has been incorporated into these vaccines.

A number of studies have been conducted using DNA vaccines encoding the MV-H protein. The immune responses generated have been of varying success. Cardoso et al. (1996) demonstrated that intramuscular inoculation of BALB/c mice with a secreted form of plasmid DNA encoding the H protein induced a class I-restricted CTL response and IgG1 antibody production (consistent with a T_{H}2-type response). Furthermore, antibody responses were not increased by multiple inoculations. In contrast, Yang et al. (1997) found that neutralizing antibody titres increased 2- to 4-fold in BALB/c mice following repeated gene-gun inoculations. In addition, these titres were better than those raised by the LAV. When similar plasmid constructs were used for macaque vaccination, however, antibody levels were found to be 100-fold lower than those elicited by a single dose of the LAV (Polack et al., 2000). Such studies highlight the dependence of an appropriate immune response on the number and route of administrations used in each particular animal model.

Bacterial and viral antigens have been expressed in transgenic plants and transiently from plant viral vectors. Antigens from both sources retain their native immunogenic properties and are able to induce neutralizing and protective antibodies in mice (Haq et al., 1995; Mason et al., 1996; Arakawa et al., 1998; Tacket et al., 1998; Wigdorovitz et al., 1999A & B). Systemic and mucosal immune responses have also been induced in human volunteers feed raw potato tubers expressing the binding subunit of the E. coli heat labile enterotoxin (LT-B) (Tacket et al. 1998). The serum antibodies produced by these volunteers were able to neutralize E. coli heat labile enterotoxin (LT) in vitro. Thus, the current data demonstrates that oral vaccination with plant-derived antigens can evoke a protective immune response.

The present invention provides an alternate strategy for inducing an immune response to an antigen in a subject. Also provided are transgenic plants expressing an antigen derived from the measles virus.

### SUMMARY OF THE INVENTION

In a first aspect, the present invention provides a method for inducing an immune response to an antigen in a subject, the method comprising administering to the subject DNA encoding the antigen, and subsequently orally administering to the subject a composition comprising transgenic material, wherein the transgenic material comprises a DNA molecule encoding the antigen such that the antigen is expressed in the transgenic material.

In a preferred embodiment of the present invention the composition further comprises a mucosal adjuvant, preferably cholera toxin β-subunits.

It is also preferred that the antigen is expressed in the transgenic material as a fusion protein. In particular it is preferred the fusion protein comprises the antigen C-terminally fused to the amino acid sequence SEKDEL (SEQ ID NO:1).

The transgenic material is preferably a transgenic plant such as a fruit or vegetable. It is preferred that the transgenic plant is selected from the group consisting of; tobacco, lettuce, rice and bananas.

In a further preferred embodiment of the present invention, the antigen is selected from the group consisting of viral antigens, parasitic antigens and bacterial antigens, preferably measles virus, the human immunodeficiency virus, or Plasmodium sp. It is preferred that the antigen is the measles virus H or F protein, or fragments thereof, preferably the measles H protein.

In a still further preferred embodiment the DNA encoding the antigen is administered to the subject on at least two occasions and the composition comprising transgenic material is orally administered to the subject on at least two occasions. More preferably, the DNA encoding the antigen is administered to the subject on a single occasion and the composition comprising transgenic material is orally administered to the subject on a single occasion.

In a second aspect the present invention provides a transgenic plant, the plant having been transformed with a DNA molecule, the DNA molecule comprising a sequence encoding a measles virus antigen such that the plant expresses the measles virus antigen.

In a preferred embodiment of this aspect of the invention, the DNA molecule encodes a fusion protein, preferably comprising the measles antigen C-terminally fused to the amino acid sequence SEKDEL.

In a further preferred embodiment the measles antigen is the measles H protein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention will hereinafter be described by way of the following non-limiting Figures and Examples.

### BRIEF DESCRIPTION OF THE ACCOMPANYING DRAWINGS

FIG. 1: Plant transformation vector constructs for expression of MV-H protein in tobacco. The T-DNA region inserted into the plant genome contains the nopaline synthase expression cassette (KanR),which confers kanamycin resistance on transformed cells, and the MV-H protein expression cassette. The MV-H protein expression cassette comprises a cauliflower mosaic virus 35S promoter (35S-Pro) fused to a tobacco etch virus 5′-untranslated region (TEV) and cauliflower mosaic virus terminator sequence (35S-Ter). The pBinH/KDEL and pBinSP/H/KDEL constructs contain an SEKDEL peptide sequence (KDEL) fused to the C-terminal end of the H protein for retention in the endoplasmic reticulum. The pBinSP/H/KDEL construct also contains a plant signal peptide (SP) fused to the N-terminal end of the H protein.

FIG. 2: Transgene expression and production of recombinant MV-H protein in transgenic tobacco. (A) Northern blot comparing the level of MV-H gene expression of the six highest expressing T₀ transgenic tobacco lines obtained for each MV-H construct. Each lane contained 10 μg of total RNA and was probed with a ³²P-labeled MV-H cDNA probe. (B) EUSA analysis of MV-H protein expression in each of the T₀ transgenic tobacco lines shown in (A) detected with a rabbit anti-measles polyclonal antibody. Four independent control transgenic lines transformed with a pBin construct lacking the MV-H gene, were included in analyses.

FIG. 3: Detection of MV-H protein in pBinH/KDEL T₁ transgenic lines. Selected kanamycin resistant progeny from the three highest T₀ expressing lines (8B, 12C and 39H) were analysed for MV-H protein expression using ELISA. The analysis was performed using either a rabbit anti-measles polyclonal antibody or MV-positive human serum. Control extract is from a transgenic tobacco line transformed with a pBin construct lacking the MV-H gene.

FIG. 4: Immune response in mice following intraperitoneal (IP) immunization with transgenic plant extracts. Five mice were immunized with leaf extract from pBinH/KDEL T₁ transgenic line 8B or a pBin control transgenic line. IP immunizations were delivered on days 0, 14 and 49 with serum collected on days 28 and 84. (A) MV-specific serum IgG. Control serum is the mean value obtained from 3-4 naïve mice. (B) MV neutralization activity of serum IgG from day 84. MV-H (●), control (∘).

FIG. 5: Immune response in mice following gavage with transgenic plant extracts. (A) Mouse serum neutralization titres following gavage. Sera collected 49 days after initial treatment were pooled and the neutralizing ability against MV assessed in plaque-reduction neutralization (PRN) assays. Naïve (♦), 2g MV-H+CT-CTB (▴), and 2g control+CT-CTB (▪). (B) MV-specific secretory IgA in faecal isolates collected 28 days after initial gavage.

FIG. 6: Serum MV neutralization (PRN) titres following DNA vaccination of mice. Sera collected 0, 15, 43 and 140 days after DNA vaccination were pooled. Naïve (♦), 2g MV-H+CT-CTB (▴), and 2g control+CT-CTB (▪).

FIG. 7: MV-specific serum IgG titres following DNA-oral prime boost vaccination. Serum IgG titres were determined by ELISA on pooled sera from 0, 21 (pre-boost) and 49 days (post-boost). (A) MV-specific serum IgG titres for mice immunized with MV-H DNA and boosted with MV-H (-▴-), or control (-▪-) plant extracts. (B) MV-specific serum IgG titres for mice immunized with control DNA and boosted with MV-H (-▴-), or control (-▪-) plant extracts. (C) Actual IgG titres represented in A and B.

FIG. 8: Serum MV neutralization (PRN) titres following DNA-oral prime boost vaccination of mice. Neutralization titres were determined using pooled sera from 0, 21 (pre-boost) and 49 days (post-boost). (A) Neutralization titre for mice immunized with MV-H DNA and boosted with MV-H (-▴-), or control (-▪-) plant extracts. (B) Neutralization titre for mice immunized with control DNA and boosted with MV-H (-▴-), or control (-▪-) plant extracts. (C) Actual neutralization titres represented in A and B.

### DETAILED DESCRIPTION OF THE PREFERRED EMBODIMENTS

Unless otherwise indicated, the recombinant DNA techniques utilized in the present invention are standard procedures, well known to those skilled in the art. Such techniques are described and explained throughout the literature in sources such as, J. Perbal, A Practical Guide to Molecular Cloning, John Wiley and Sons (1984); J. Sambrook et al., Molecular Cloning: A Laboratory Manual, Cold Spring Harbour Laboratory Press (1989); T.A. Brown (editor), Essential Molecular Biology: A Practical Approach, Volumes 1 and 2, IRL Press (1991); D. M. Glover and B. D. Hames (editors), DNA Cloning: A Practical Approach, Volumes 1-4, IRL Press (1995 and 1996); and F. M. Ausubel et al. (editors), Current Protocols in Molecular Biology, Greene Pub. Associates and Wiley-Interscience (1988, including all updates until present) and are incorporated herein by reference.

DNA vaccination involves the direct in vivo introduction of DNA encoding an antigen into tissues of a subject for expression of the antigen by the cells of the subject's tissue. Such vaccines are termed herein “DNA vaccines” or “nucleic acid-based vaccines.” DNA vaccines are described in U.S. Pat. No. 5,939,400, U.S. Pat. No. 6,110,898, WO 95/20660 and WO 93/19183, the disclosures of which are hereby incorporated by reference in their entireties. The ability of directly injected DNA that encodes an antigen to elicit a protective immune response has been demonstrated in numerous experimental systems (see, for example, Conry et al., 1994; Cardoso et al., 1996; Cox et al., 1993; Davis et al., 1993; Sedegah et al., 1994; Montgomery et al., 1993; Ulmer et al., 1993; Wang et al., 1993; Xiang et al., 1994; Yang et al., 1997).

To date, most DNA vaccines in mammalian systems have relied upon viral promoters derived from cytomegalovirus (CMV). These have had good efficiency in both muscle and sidn inoculation in a number of mammalian species. A factor known to affect the immune response elicited by DNA immunization is the method of DNA delivery, for example, parenteral routes can yield low rates of gene transfer and produce considerable variability of gene expression (Montgomery et al., 1993). High-velocity inoculation of plasmids, using a gene-gun, enhanced the immune responses of mice (Fynan et al., 1993; Eisenbraun et al., 1993), presumably because of a greater efficiency of DNA transfection and more effective antigen presentation by dendritic cells. Vectors containing the nucleic acid-based vaccine of the invention may also be introduced into the desired host by other methods known in the art, e.g., transfection, electroporation, microinjection, transduction, cell fusion, DEAE dextran, calcium phosphate precipitation, lipofection (lysosome fusion), or a DNA vector transporter.

“Transgenic material” of the present invention refers to any substance of biological origin that has been genetically engineered such that it produces the antigen. Preferably, the transgenic material is a transgenic plant.

The orally administered composition can be administered by the consumption of a foodstuff, where the edible part of the transgenic material is used as a dietary component while the antigen is provided to the subject in the process.

The present invention allows for the production of not only a single antigen in the DNA vaccine and/or the transgenic material but also allows for a plurality of antigens.

DNA sequences of multiple antigenic proteins can be included in the expression vector used for transformation of an organism, thereby causing the expression of multiple antigenic amino acid sequences in one transgenic organism. Alternatively, an organism may be sequentially or simultaneously transformed with a series of expression vectors, each of which contains DNA segments encoding one or more antigenic proteins. For example, there are five or six different types of influenza, each requiring a different vaccine. Transgenic material expressing multiple antigenic protein sequences can simultaneously boost an immune response to more than one of these strains, thereby giving disease immunity even though the most prevalent strain is not known in advance.

Plants which are preferably used in the practice of the present invention include any dicotyledon and monocotyledon which is edible in part or in whole by a human or an animal such as, but not limited to, carrot, potato, apple, soybean, rice, corn, berries such as strawberries and raspberries, banana and other such edible varieties. It is particularly advantageous in certain disease prevention for human infants to produce a vaccine in a juice for ease of oral administration to humans such as tomato juice, soy bean milk, carrot juice, or a juice made from a variety of berry types. Other foodstuffs for easy consumption include dried fruit.

Several techniques exist for introducing foreign genetic material into a plant cell, and for obtaining plants that stably maintain and express the introduced gene. Such techniques include acceleration of genetic material coated onto microparticles directly into cells (see, for example, U.S. Pat. No. 4,945,050 and U.S. Pat. No. 5,141,131). Plants may be transformed using Agrobacterium technology (see, for example, U.S. Pat. No. 5,177,010, U.S. Pat. No. 5,104,310, U.S. Pat. No. 5,004,863, U.S. Pat. No. 5,159,135). Electroporation technology has also been used to transform plants (see, for example, WO 87/06614, U.S. Pat. No. 5,472,869, 5,384,253, WO 92/09696 and WO 93/21335). Each of these references are incorporated herein by reference. In addition to numerous technologies for transforming plants, the type of tissue which is contacted with the foreign genes may vary as well. Such tissue would include but would not be limited to embryogenic tissue, callus tissue type I and II, hypocotyl, meristem, and the like. Almost all plant tissues may be transformed during development and/or differentiation using appropriate techniques described herein.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′4 and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

Examples of plant promoters include, but are not limited to ribulose-1,6-bisphosphate carboxylase small subunit, beta-conglycinin promoter, phaseolin promoter, ADH promoter, heat-shock promoters and tissue specific promoters. Promoters may also contain certain enhancer sequence elements that may improve the transcription efficiency. Typical enhancers include but are not limited to Adh-intron 1 and Adh-intron 6.

Constitutive promoters direct continuous gene expression in all cells types and at all times (e.g., actin, ubiquitin, CaMV 35S). Tissue specific promoters are responsible for gene expression in specific cell or tissue types, such as the leaves or seeds (e.g., zein, oleosin, napin, ACP, globulin and the like) and these promoters may also be used. Promoters may also be active during a certain stage of the plants' development as well as active in plant tissues and organs. Examples of such promoters include but are not limited to pollen-specific, embryo specific, corn silk specific, cotton fiber specific, root specific, seed endosperm specific promoters and the like.

Under certain circumstances it may be desirable to use an inducible promoter. An inducible promoter is responsible for expression of genes in response to a specific signal, such as: physical stimulus (heat shock genes); light (RUBP carboxylase); hormone (Em); metabolites; and stress. Other desirable transcription and translation elements that function in plants may be used.

In addition to plant promoters, promoters from a variety of sources can be used efficiently in plant cells to express foreign genes. For example, promoters of bacterial origin, such as the octopine synthase promoter, the nopaline synthase promoter, the mannopine synthase promoter; promoters of viral origin, such as the cauliflower mosaic virus (35S and 19S) and the like may be used.

A number of plant-derived edible vaccines are currently being developed for both animal and human pathogens (Hood and Jilka, 1999). Immune responses have also resulted from oral immunization with transgenic plants producing virus-like particles (VLPs), or chimeric plant viruses displaying antigenic epitopes (Mason et al., 1996; Modelska et al., 1998; Kapustra et al., 1999; Brennan et al., 1999). It has been suggested that the particulate form of these VLPs or chimeric viruses may result in greater stability of the antigen in the stomach, effectively increasing the amount of antigen available for uptake in the gut (Mason et al. 1996, Modelska et al. 1998).

Mutant and variant forms of the DNA sequences encoding for a particular antigen may also be utilized in this invention. For example, expression vectors may contain DNA coding sequences which are altered so as to change one or more amino acid residues in the antigen expressed in the transgenic material, thereby altering the antigenicity of the expressed protein. Expression vectors containing a DNA sequence encoding only a portion of an antigenic protein as either a smaller peptide or as a component of a new chimeric fusion protein are also included in this invention.

The present invention can be used to produce an immune response in animals other than humans. Diseases such as: canine distemper, rabies, canine hepatitis, parvovirus, and feline leukemia may be controlled with proper immunization of pets. Viral vaccines for diseases such as: Newcastle, Rinderpest, hog cholera, blue tongue and foot-mouth can control disease outbreaks in production animal populations, thereby avoiding large economic losses from disease deaths. Prevention of bacterial diseases in production animals such as: brucellosis, fowl cholera, anthrax and black leg through the use of vaccines has existed for many years. The transgenic material used in the methods of the present invention may be incorporated into the feed of animals.

A “mucosal adjuvant” is a compound which non-specifically stimulates or enhances a mucosal immune response (e.g., production of IgA antibodies). Administration of a mucosal adjuvant in a composition facilitates the induction of a mucosal immune response to the immunogenic compound.

The mucosal adjuvant may be any mucosal adjuvant known in the art which is appropriate for human or animal use. For example, the mucosal adjuvant may be cholera toxin (CT), enterotoxigenic E. Coli heat-labile toxin (LT), or a derivative, subunit, or fragment of CT or LT which retains adjuvanticity. Preferably, the mucosal adjuvant is cholera toxin β-subunits. The mucosal adjuvant is co-administered with the composition comprising transgenic material in an amount effective to elicit or enhance a mucosal immune response. The suitable amount of adjuvant may be determined by standard methods by one skilled in the art. Preferably, the adjuvant is present at a ratio of 1 part adjuvant to 10 parts composition comprising the transgenic material.

In the present invention, the antigen can be expressed in the transgenic material as a fusion protein. Typically, the additional amino acid sequence will extend from the C-terminus and/or the N-terminus of the antigen. Preferably, the fusion protein results in a higher immune response when compared to when the antigen not expressed as a fusion protein. It is also preferred that the fusion protein comprise at least two antigens from the same or different native protein. In the latter instance, the different antigens can be from different organisms, providing immune protection against a number of pathogens.

### EXAMPLE

Experimental Protocol

Construction of Transgenic Tobacco Plants Producing H Protein

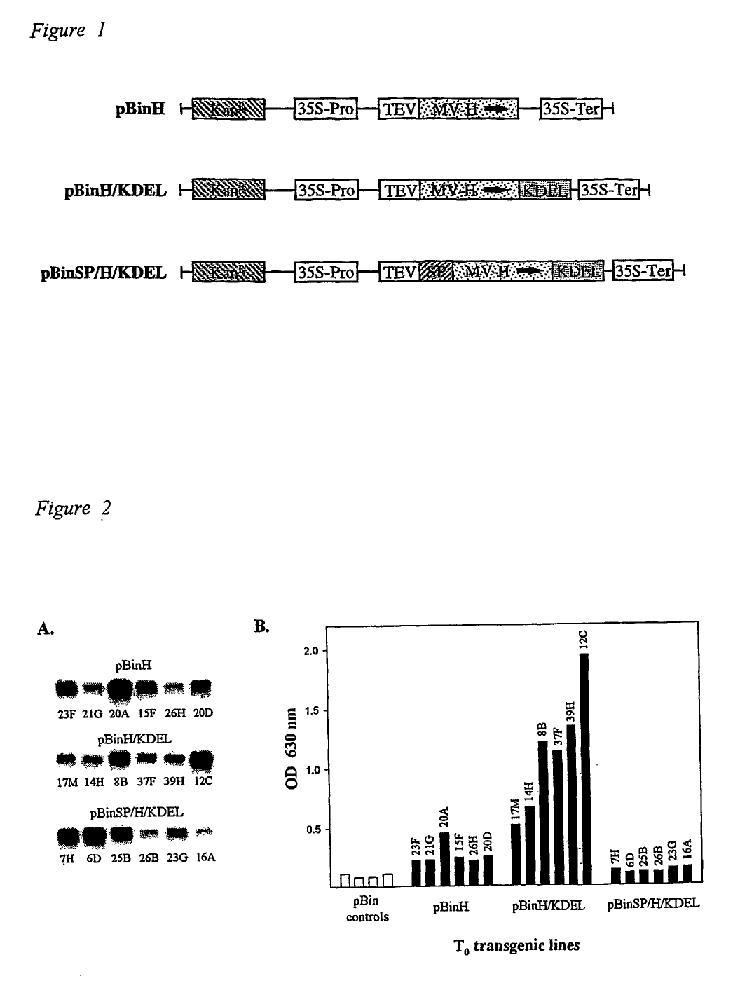

Three constructs were generated for the expression of MV-H protein in tobacco plants (FIG. 1) (a) pBinH—H protein alone, (b) pBinDEL—addition of a C-terminal endoplasmic reticulum (ER)-retention sequence and (c) pBinSP/H/KDEL—addition of both an N-terminal plant signal peptide and a C-terminal ER-retention sequence.

To produce these constructs a 1.8 kb EcoRI/BamHI fragment encompassing the open reading frame of the MV-H gene (Edmonston strain; GenBank accession no. X16565) was obtained from plasmid pBS-HA Johns Hopidns Hospital, Baltimore). Using the Altered Sites kit (Promega) an NcoI site was introduced into the 5′-end of the H gene. The NcoI site was created around the existing initiation codon by mutating the first nucleotide of the second codon from T to C. This also altered the second amino acid of the H protein from serine to alanine. The NcoI/BamHI fragment containing the N-terminal modified H gene was then transferred into the plant expression vector pRTL2 (Restrepo et al., 1990) to give pRTL2-H.

A second H-protein construct containing the NcoI site described above and an endoplasmic reticulum-retention sequence SEKDEL (Munro and Pelham, 1987) was also engineered. A XhoI site was introduced into the C-terminus of the H gene immediately upstream of the stop codon and BamHI site using the Altered Sites kit (Promega). This allowed a double-stranded oligonucleotide encoding the SEKDEL sequence to be ligated between the XhoI and BamHI sites creating an in-frame fusion with the C-terminal end of the H protein. The SEKDEL oligonucleotide was produced by annealing the following complementary sequences: 5′-TCGATCTCTGAGAAAGATGAGCTATGAGGG-3′ (SEQ ID NO:2) and 5′-GATCCCCTCATAGCTCAT CTTTCTCAGAGA-3′ (SEQ ID NO:3). The C-terminal sequence of the modified H protein was altered from TNRR* (SEQ ID NO:4) to TNLQSEKDEL* (SEQ ID NO: 5). The H/KDEL fragment was then cloned into pRTL2 to give pRTL2-H/KDEL.

In the third construct, the signal peptide (SP) of the tobacco Pr1α gene (Hammond-Kosack et al. 1994) was cloned into the NcoI site of pRTL2-H/KDEL upstream of, and in frame with, the H protein. The 107 bp SP fragment was amplified by PCR from the plasmid SLJ6069 (Sainsbury Laboratory, JIC, Norwich, UK) using the oligonucleotides: 5′-GCGCCATGGGATTTGTTCTCTTT-3′ (SEQ ID NO: 6) and 5′-TATCCATGGGCCCGGCACGGCAAGAGTGGGATAT-3′ (SEQ ID NO:7). This clone was designated pRTL2-SP/H/KDEL.

Following verification of modifications by sequence analysis, the expression cassettes of pRTL2-H, pRTL2-H/KDEL, and pRTL2-SP/H/KDEL were transferred into the binary vector pBin19 (Bevan, 1984) to produce pBinH, pBinH/KDEL and pBinSP/H/KDEL, respectively (FIG. 1).

These three constructs were then electroporated into Agrobacterium tumefaciens strain LBA 4404 and used for transformation of tobacco (Nicotiana tabacum var Samsun) using the leaf disc method as described by Horsch et al. (1985).

Transgene Expression Analysis

Total RNA was extracted from 150 mg leaf samples of in vitro transgenic tobacco plants in 0.1M Tris, 0.1M NaCl, 10 mM EDTA, 1% SDS, 1% β-mercaptoethanol, pH 9.0 by extracting twice with an equal volume of phenol and once with equal volume of phenol:chloroform:isoamyl alcohol (25:24:1 v/v). The final aqueous phase was mixed with 0.1 volume of sodium acetate (pH 5.0) and 2.5 volumes of cold 100% ethanol, incubated at −20° C. for 30 min and nucleic acid pelleted by centrifugation at 13,000 g for 10 min. The pellet was rinsed with cold 70% ethanol, dried and resuspended in 25 μl of sterile water. RNA was analysed by northern blot using a ³²P-labelled MV-H cDNA probe.

Detection of MV-H Protein in Transgenic Tobacco by ELISA

Tobacco leaves (50 mg) were frozen in liquid nitrogen and ground to a fine powder in a 1.5 ml eppendorf. Five volumes of chilled extraction buffer (PBS containing 100 mM ascorbic acid, 20 mM EDTA, 0.1% Tween-20 and 1 mM PMSF, pH 7.4) was added and the extract vortexed for 15 s. The extract was then centrifuged at 23,000 g for 15 min at 4° C., the supernatant collected and glycerol added to a final concentration of 16% before snap freezing in liquid nitrogen and storage at −70° C.

Plant extracts were diluted in 0.1M carbonate buffer (pH 9.6) and were coated onto ELISA plates at 4° C. overnight All further incubations were at 37° C. for 1 hour. Following a blocking step with 2.5% sldm milk the MV-H protein was detected with a rabbit polyclonal anti-measles antibody (CDC, Atlanta) diluted 1/4000. Anti-rabbit horseradish peroxidase conjugate (Boehringer Mannheim) diluted 1/8000 was used as the secondary antibody. The plates were developed with TMB (3,3′,5,5′-tetramethylbenzidine) substrate for 30-60 min and read at 630 nm.

Preparation of Antigen from Transgenic Plants

Recently expanded leaves from glasshouse grown plants of the pBinH/KDEL transgenic line 8B, or transgenic tobacco lacking the MV-H gene, were harvested and stored at −35° C. All subsequent steps were performed on ice or at 4° C. Frozen tobacco leaves were powdered in a coffee grinder and mixed with 2.5 volumes of chilled extraction buffer (described above). The extract was filtered through 2 layers of miracloth, centrifuged at 100 g for 5 min and the supernatant centrifuged again at 32,600 g for 60 min. Glycerol was added to the pellet to a final concentration of 16% allowing the extracts to be stored at −70° C. Extracts ranged in concentration from 3.2g/ml to 4.5g/ml.

The supernatant from the 32,600g spin was further purified. Proteins precipitated from the supernatant between 25% and 50% ammonium sulphate (AS) were resuspended in phosphate buffered saline (PBS) containing 10 mM ascorbic acid, and applied to PD-10 columns (Amersham Pharmacia Biotech, Uppsala, Sweden) pre-equilibrated with PBS. The protein fraction was eluted in PBS, glycerol was added to a final concentration of 16% allowing the extracts to be stored at −70° C.

A mucosal adjuvant consisting of 2 μg of cholera toxin (CT) and 10 μg of cholera toxin B subunit (CTB) (Sigma, USA) was added to plant aliquots immediately prior to gavage. Gavage was performed using an 8 cm gavage needle attached to a 1 ml Tuberculin syringe. The gavage needle was inserted down the oesophagus of anaesthetized animals into the stomach, where 0.4g, 1 g, 2 g or 4 g of plant material was injected. Mice were studied for signs of tracheal or nasal obstruction until fully recovered from anaesthetic.

Laboratory Mice and Cell Lines

Adult female Balb/c mice, between 18-25 g (approximately 8 weeks old), were purchased from Animal Research Centre, Western Australia, and were maintained in the University Animal House. Rhesus monkey kidney cells (RMK cells) were grown as monolayers at 37° C. in RPMI 1640 medium (Trace, Biosciences Ltd, Australia) supplemented with 10% fetal calf serum (FCS) (Trace) in a 5% CO₂ atmosphere.

Construction and Vaccination of MV-H DNA

A high copy pCI plasmid vector (Promega, USA) incorporating a human cytomegalovirus (CMV) immediate-late enhancer/promoter, ampicillin resistance and the SV40 late polyadenylation signal was used for vaccine production. Two DNA vaccine constructs were prepared. One containing the extracellular domain of the measles virus H gene (MV-H), and a control construct containing the ovalbumin gene.

A 1 ml Insulin needle (Becton Dickinson, USA) was used to inject 25 or 50 μg of DNA solution into both quadriceps of each mouse.

Collection of Mouse Samples

Blood was collected by intraocular bleeding or cardiac puncture, once blood had clotted serum was recovered by centrifugation (7100 g, 6 min).

Faeces were collected into eppendorfs pre-blocked with 1% BSA. 1 ml of 0.1% BSA+0.15 mM PMSF solution in PBS was added per 100 mg of faeces. Following overnight incubation at 4° C., solid material was disrupted by vortexing then centrifuged (25,000 g, 6 min). The supernatant was stored at −20° C. in pre-blocked eppendorfs.

To collect saliva samples anaesthetized mice were injected with 200 μl of 20 μg/ml carbachol in PBS to induce salivation.

Bronchoalveolar fluid was collected from killed mice. The throat region was exposed and muscle tissue surrounding the trachea removed. A small hole was made in the trachea and a lavage tip attached to a 1 ml Tuberculin syringe containing 0.4 ml of wash solution (1% v/v foetal calf serum in PBS) was inserted. After dispensing wash solution into the lungs, a 10 second rib-cage massage was performed prior to retraction of the syringe plunger and the extraction of lung fluid. Two more washes were performed using 0.3 ml of wash solution.

Detection of MV-Specific Antibodies

Enzygnost measles-coated plates (Dade-Behring, Germany), containing simian kidney cells infected with MV, were used for detection of anti-MV antibody in mouse samples. MV-specific antibodies were detected with peroxidase-conjugated goat anti-mouse IgG followed by tetramethyl-bromide (TMB) substrate.

IgG-typing was performed using alkaline phosphatase (AP)-conjugated anti-mouse IgG1 or AP-conjugated anti-mouse IgG2a and p-Nitrophenyl phosphate (pNPP) substrate.

Mouse serum, salivary, BAL and faecal samples were assayed for the presence of IgA using AP-conjugated goat anti-mouse IgA with pNPP substrate.

Plaque Reduction Neutralization Assay

The plaque reduction neutralization (PRN) titre is the reciprocal of the serum dilution capable of preventing 50% plaque formation by wild-type MV. The Edmonston strain of MV was used for this assay.

Four-fold dilutions of heat inactivated sera were prepared in supplemented RPMI (¼ to 1/4096) and added to an equal volume of MV (200pfu/100 μl). This serum/virus suspension was incubated at 37° C. for 90 minutes before addition to 24-well, flat-bottomed plates containing 80% confluent RMK cells. Following a 90 minute incubation at 37° C. 1 ml/well of supplemented RPMI medium was added and plates were incubated at 37° C. in a humidified atmosphere of 5% CO₂ for 72 hours.

Growth medium was removed and cells were fixed and permeabilised with 1 ml/well of 10% formaldehyde with 0.1% Triton-X 100 in PBS for 20 minutes at RT. Plates were blocked with goat serum and anti-MV IgG positive human serum was added. Anti-MV human sera was detected with FITC-conjugated anti-human IgG and fluorescing cells were examined using a Leitz fluovert inverted fluorescent microscope. Each cluster of fluorescing, infected cells was counted as one pfu. The serum dilution capable of preventing 50% plaque formation was generated according to the Karber formula.

Results

Tansgenic Tobacco Plants Producing MV-H Protein

A 1.8 kb fragment encompassing the coding region of the MV hemagglutinin (H) gene (Edmonston strain) was cloned into a plant expression cassette (FIG. 1). To compare the effect of intracellular targeting on antigen yield, two additional clones were constructed, with a C-terminal SEKDEL sequence, coding for retention in the ER (pBinH/KDEL; Munro and Pelham 1987), and an authentic N-terminal plant signal peptide (pBinSP/H/KDEL; Hammond-Kosack et al., 1994).

A total of 90 primary transformant (T₀) lines were obtained which showed detectable levels of MV-H gene expression by northern blot analysis (data not shown). A comparison of the six highest expressing lines for each construct are shown in FIG. 2A. Transgene expression was similar for all three constructs. The selected high expressors shown in FIG. 2A were further analysed for level of recombinant MV-H protein by ELISA using a rabbit anti-measles polyclonal antibody (FIG. 2B). Plants transformed with the pBinH construct produced small quantities of recombinant MV-H protein. However, addition of the C-terminal KDEL sequence resulted in much higher levels of MV-H protein accumulation in plants transformed with the pBinH/KDEL construct. Interestingly, addition of the Pria plant signal peptide appeared to inhibit MV-H protein production in pBinSP/H/KDEL lines relative to the H/KDEL transgenic lines. For tobacco lines containing constructs pBinH and pBinH/KDEL, there appeared to be a reasonable correlation between transgene expression level and MV-H protein production (compare FIGS. 2A & 2B).

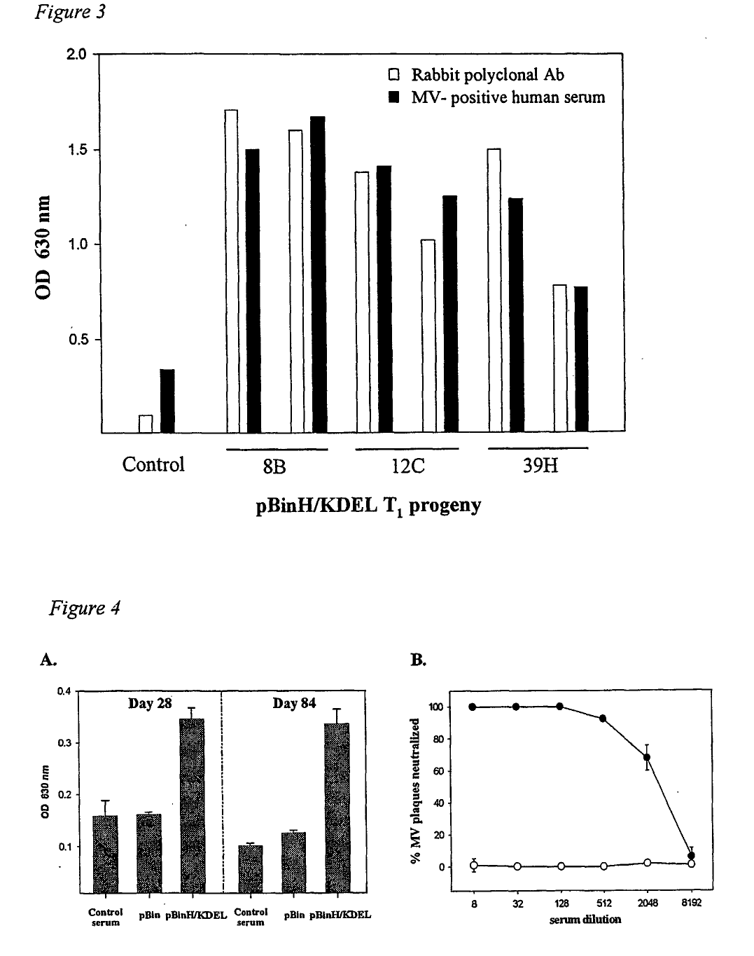

Seed was collected from the pBinH/KDEL T₀ transgenic lines showing the highest levels of H production (12C, 8B & 39H), germinated on kanamycin and re-assayed for MV-H protein production. ELISA analysis using the rabbit anti-measles polyclonal antiserum showed that the introduced MV-H transgene was stably inherited in the T₁ progeny (FIG. 3). Recombinant MV-H protein could also be detected in leaf extracts of pBinH/KDEL T₁ progeny by human serum (FIG. 3). This serum was obtained from a subject with a history of wild-type measles infection, who had tested positive for measles antibodies by ELISA. The human serum detected similar quantities of MV-H protein in T₁ plants as the rabbit anti-measles polyclonal antiserum (FIG. 3), confirming that the plant-derived MV-H protein retained at least some of the antigenic regions present in the native MV-H protein.

Further evidence of the authentic antigenicity of the recombinant MV-H protein was its positive reaction with two out of three MV-H protein monoclonal antibodies as tested by indirect ELISA. MAb-366 detected MV-H protein in extracts of pBinH/KDEL 8B (T₁) line with absorbance readings ranging from 0.392 to 0.420, compared to 0.018 to 0.019 for extracts from pBin control transgenic. The response of MAb-CV4 provided absorbance values ranging from 0.063 to 0.065 for the pBinH/KDEL extracts, compared to −0.005 to −0.001 for control transgenic extracts.

Intraperitoneal Vaccination with Plant-Derived MV-H Protein Induces MV Neutralizing Antibodies

To determine the immunogenicity of the plant-derived MV-H protein groups of BAL3/c mice were inoculated intraperitoneally with AS-purified plant extract from MV-H or control transgenic plants. Mice were inoculated on day 0, 14 and 49 and serum was collected on day 28 and 84. Significantly more MV-specific IgG was detected in mice vaccinated with plant-derived MV-H than in mice inoculated with control plant extract (P<0.01) (FIG. 4A). The MV-specific IgG was able to neutralize wild-type MV in vitro (FIG. 4B). These results demonstrate that plant-derived MV-H protein is immunogenic when administered intraperitoneally.

Oral Vaccination with Plant-Derived MV-H Protein Induces Neutralizing Antibodies and sIgA

Mice gavaged with either AS-purified MV-H or pellet MV-H extract have developed neutralizing antibodies to wild-type MV, details of one of these experiments are given below.

Groups of three mice were given 1 g, 2 g or 4 g of plant extract containing the mucosal adjuvant CT-CTB by gavage on days 0, 7, 14, 21 and 35. Sera were collected on days 0, 7, 14, 21, 28, 49 and 78 and faecal isolates obtained on days 0 and 28. MV-specific serum IgG was only detected in groups that received 2 g or 4 g of MV-H plant extract. The serum IgG responses persisted for at least 78 days in mice gavaged with 2 g of extract, but for only 49 days in mice gavaged with 4 g of extract, with maximum titres of 2187 and 9 respectively. The lower response to 4 g may be due to the increased dose to tobacco toxins also received.

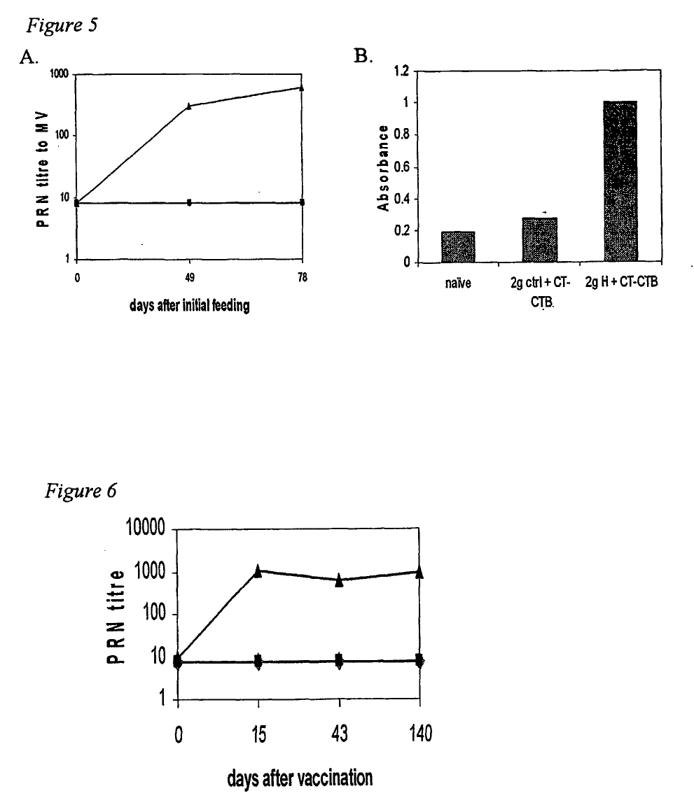

High neutralizing ability was observed in pooled sera collected from mice gavaged with 2 g of MV-H plant extract (FIG. 5A). It peaked at 78 days with a PRN titre of 600. Mice gavaged with 4 g of MV-H plant extract had a maximum neutralization titre of 150 at day 49. No neutralizing ability was detected in mice gavaged with 2 g of control plant extract.

MV-specific secretory IgA (sIgA) was detected in faecal samples from some mice gavaged with 2g of MV-H plant extract (FIG. 5B). This is a particularly important result as mucosal immunity is the first line of defense against airborne pathogens such as measles.

Vaccination with MV-H DNA Constructs Induces MV-Neutralizing Antibodies

Groups of five mice were injected with 100 μg of MV-H DNA, or ovalbumin DNA (control) on day 0. Sera was collected on days 0, 15, 43 and 140, and faecal samples were obtained on days 0, 7, 14 and 21. Ten days after vaccination an increase in MV-specific IgG was only observed in the experimental group that received MV-H DNA. High serum IgG levels were maintained from day 20 to day 43, with a maximum titre of 729. In contrast to mice immunized with control DNA, which produced no MV-specific immune response, serum IgG from mice primed with MV-H DNA was able to neutralize wild-type MV in vitro (FIG. 6). A neutralization titre of 900 was recorded at day 140, suggesting that the immune response is persistent High titres of MV-neutralizing antibodies have previously been raised using MV-H DNA vaccines in mice (Yang et al. 1997, Polack et al. 2000), however some studies suggest that maternal antibodies many interfere with vaccine efficiency (Schlereth et al. 2000).

The predominant isotype present in mice immunized with MV-H DNA was IgG1, indicating a T_{H}2-type response. While intramuscular DNA vaccines are generally associated with T_{H}1-type responses, T_{H}2 dominated responses have been reported to occur in response to intramuscular DNA vaccination with a secreted form of measles H protein and a secreted hemagglutinin-based influenza DNA vaccine (Cardoso et al. 1996, Deliyannis et al. 2000). It is possible that this switching of IgG isotypes is due to a difference in antigen presentation when the encoded antigen is released from, rather than retained within, transfected cells, although there are no conclusive data to account for these differences.

No MV-specific serum or secretory IgA was detected in any DNA immunized group.

Oral Delivery of MV-H Protein Following MV-H DNA Vaccine Boosts Serum IgG Titres

Mice were primed with 50 μg of MV-H or control DNA on day 0. On days 21, 28, 35 and 42, these mice were boosted with 2g of either control or H protein plant extract, administered with CT-CTB. Sera were collected on days 0, 21 (pre-boost), and 49 (post-boost), and faecal isolates were obtained weekly until day 49. Salivary and bronchoalveolar lavage (BAL) samples were collected on day 49. Five mice were used per treatment.

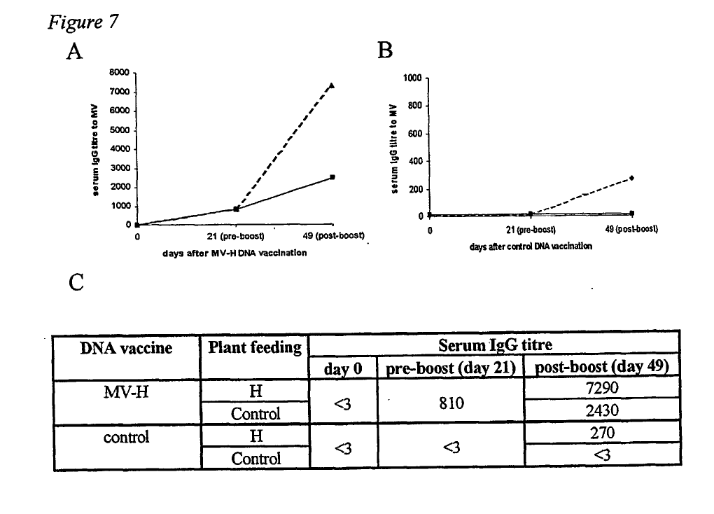

MV-specific serum IgG titres were determined for pre-boost and post-boost pooled sera (FIG. 7). Mice primed with MV-H DNA, produced MV-specific IgG, but mice given control DNA did not. The titre of the MV-H DNA IgG response was increased three-fold following gavage with MV-H plant extract. MV-H DNA primed mice boosted with control plant extracts also had higher post-boost IgG titres. However the absence of MV-specific serum IgG in mice primed with control DNA and boosted with control plant extract indicates that this is due to a continuing response to the MV-H DNA vaccine and not to the control plant extract. Delivery of the MV-H DNA vaccine followed by an oral MV-H plant boost resulted in higher serum IgG titres than either DNA vaccination or oral plant vaccination alone (MV-H DNA-control plant and control DNA-MV-H plant respectively).

Oral Delivery of MV-H Protein Following MV-H DNA Vaccine Boosts Neutralization Titres

Neutralization assays were performed on pooled sera collected prior to DNA vaccination (day 0), immediately before boosting with plant extracts (day 21) and 1 week after the final plant feeding (day 49) for each of the four treatment groups.

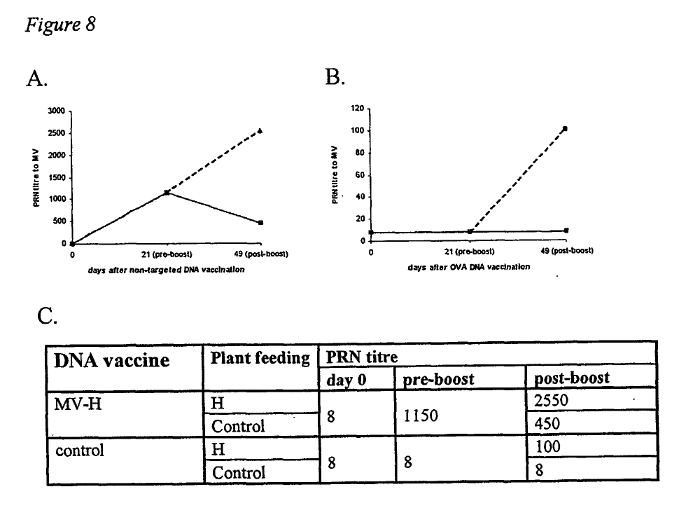

The neutralization titres exhibited similar trends to the IgG titres (FIG. 8). At day 21 (pre-boost) serum from MV-H DNA primed mice had an average neutralization titre of 1150 compared to a titre of 8 for mice primed with control DNA. Following gavage with MV-H plant extracts neutralization titres increased relative to titres for mice boosted with control plant extract (FIG. 8). The neutralization titre for MV-H DNA primed mice boosted with control plant dropped from 1150 to 450, while mice boosted with MV-H plant extract exhibited an increase in neutralization titre from 1150 to 2550. This suggests that boosting with MV-H plant extract has enhanced both the magnitude and the persistence of the immune response.

As with serum IgG titres combining the MV-H DNA vaccine and MV-H plant extract resulted in a synergistic response producing neutralization titres in excess of those recorded for either DNA or plant extract alone (FIG. 8).

The present invention demonstrates that MV-H protein can be expressed in transgenic material and that this recombinant protein is recognised by host antibodies produced in response to wild-type measles infection. Furthermore the present invention shows that mice immunized intraperitoneally, by gavage or by DNA-oral prime-boost all developed antibodies able to neutralize wild-type MV in vitro (FIGS. 4B, 5A, 8). Neutralization titres for serum IgG were greater following DNA-oral prime boost than when either DNA or plant extracts were used alone (FIG. 8). Finally, oral immunization using plant-derived MV-H protein resulted in the production of measurable levels of MV-specific sIgA (FIG. 5B).

The present study demonstrates that “DNA vaccination-oral prime-boost” vaccination strategy utilising transgenic organisms is a viable approach to new vaccines. The potential for inducing a mucosal immune response, and seroconversion in the presence of maternal antibodies are important advances of this vaccine strategy. Availability of the vaccine in an “edible” form as a constituent of a fruit or vegetable crop will also enhance vaccination coverage by providing an inexpensive and relatively heat-stable package for distribution. Such a vaccine will have the potential to enable rates of vaccination to reach the targets required for global eradication.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

### REFERENCES

Arakawa T., Chong D. K. X. and Langridge W. H. R. (1998) Nature Biotech 16:292-297.

Babbott, F. and Gordon, J. E. (1954) Prog Med Sci 228:934-938.

Bevan M. (1984) Nucl Acids Res 12:8711-8721.

Brennan F. R., Bellaby T., Helliwell S. M., Jones T. D., Kamstrup S., Dalsgaard K., Flock J-I. and Hamilton W. D. O. (1999) J Virol 73:930-938.

Cardoso A. I., Blixenkrone-Moller M., Fayolle J., Liu M., Buckland R. and Wild T. F. (1996) Immuniz Virol 225:293-299.

Conry, R. M., LoBuglio, A. F., Kantor, J., Schlom, J., Loechel, F., Moore, S. E., Sumerel, L. A., Barlow, D. L., Abrams, S. and Curiel, D. T. (1994) Cancer Res 54:1164-1168.

Cox, G. J., Zamb, T. J. and Babiuk, L. A. (1993) J Virol 67:5664-5667.

Cutts F. T. and Steinglass R. (1998) British Med J 316:765-767.

Davis, H. L., Michel, M. L. and Whalen, R. G. (1993) Hum Mol Genet 2:1847-1851.

Deliyannis G., Boyle J. S., Brady J. L., Brown L. E. and Lew A. M. (2000) Proc Natl Acad Sci USA 97:6676-6680.

Eisenbraun, M. D., Fuller, D. H. and Haynes, J. R. (1993) DNA Cell Biol 12:791-797.

Fynan, E. F., Webster, R. G., Fuller, D. H., Haynes, J. R., Santoro, J. C. and Robinson, H. L. (1993) Proc Natl Acad Sci USA 90:11478-11482.

Gans H. A., Arvin A. M., Galinus J., Logan L., DeHovitz R. and Maldonado Y. (1998) J Am Med Assoc 280:527-532.

Garenne M., Leroy O., Beau J. P., and Sene I. (1991) Lancet 338:903-907.

Hammond-Kosack K. E., Harrison K. and Jones J. D. G. (1994) Proc Natl Acad Sci USA 91:10445-10449.

Haq T., Mason H. S., Clements J. D. and Arntzen C. J. (1995) Science 268:714-716.

Hood E. E. and Jilka J. M. (1999) Curr Opin Biotech 10:382-386.

Horsch, R. B., Fry, J. E., Hoffmann, N. L., Eichholtz, D., Rogers, S. G., and Fraley, R. T. (1985) Science 227:1229-1231.

Kapustra J., Modelska A., Figlerowicz M., Pniewski T., Letellier M., Lisowa O., Yusibov V., Koprowsid H., Plucieiczak A. and Legocid A. B. (1999) FASEB J 13:1796-1799.

Markowitz, L. E., Sepulveda, J., Diaz-Ortega, J. L., Valdespino, J. L., Albrecht, P., Zell, E. R., Stat, M., Stewart, J., Zarate, M. L., Bernier, R H. (1990) New England J Med 322:580-587.

Mason H. S., Ball J. M., Shi J. J., Jiang X., Estes M. K. and Arntzen C. J. (1996) Proc Natl Acad Sci USA 93:5335-5340.

Mason H S, Haq T., Clements J. D., and Arntzen C. J. (1998) Vaccine 16:1336-1343.

Modelska A., Dietzschold B., Sleysh N., Fu Z. F., Steplewski K., Hooper D. C., Koprowski H. and Yusibov V. (1998) Proc Natl Acad Sci USA 95:2481-2485.

Montgomery, D. L., Shiver, J. W., Leander, K. R., Perry, H. C., Friedman, A., Martinez, D., Ulmer, J. B., Donnelly, J. J. and Lui, M. A. (1993) DNA Cell Biol 12:777-783.

Munro S. and Pelham, H. R. B. (1987) Cell 48:899-907.

Polack F. P., Lee S. H., Permar S., Manyara E., Nousari H. G., Jeng Y., Mustafa F., Valsamalds A., Adams R. J., Robinson H. L. and Griffin D. E. (2000) Nature Med 6:776-781.

Restrepo M. A., Freed D. D. and Carrington J. C. (1990) Plant Cell 2:987-998.

Schlereth, B., Germann, P. G., terMeulen, V., and Niewiesk, S. (2000) J Gen Virol 81:1321-1325.

Sedegah, M., Hedstrom, R., Hobart, P. and Hoffman, S. L. (1994) Proc Natl Acad Sci USA 91:9866-9870.

Tacket C. O., Mason H. S., Losonsky G., Clements J. D., Levine M. M. and Arntzen C. J. (1998) Nature Med 4:607-609.

Weiss R. (1992) Science 258:546-547.

Wigdorovitz, A., Filgueira. D. M. P., Robertson, N., Carrillo, C., Sadir, A. M., Morris, T. J. and Borca, M. V. (1999A) Virology 264:85-91.

Wigdorovitz A., Carrillo C., Dus Santos M. J., Trono K., Peralta A., Gomez M. C., Rios R. D., Franzone P. M., Sadir A. M., Escribano J. M. and Borca M. V. (1999B) Virology 255:347-353.

Ulmer, J. B., Donnelly, J. J., Parker, S. E., Rhodes, G. H., Felgner, P. L., Dwarki, V. J., Gromkowski, S. H., Deck, R. R., DeWitt, G. M., Friedman, A. et al. (1993) Science 259:1745-1749.

Wang, B., Ugen, K. E., Srikantan, V., Agadjanyan, M. G., Dang, K., Refaeli, Y., Sato, A. I., Boyer, J., Williams, W. V. and Weiner, D. B. (1993) Proc Natl Acad Sci USA 90:4156-4160.

Xiang, Z. Q., Spitalnik, S., Tran, M., Wunner, W. H., Cheng, J. and Ertl, H. C. (1994) Virology 199:132-140.

Yang K., Mustafa F., Valsamakis A., Santoro J. C., Griffin D. E. and Robinson H. L. (1997) Vaccine 15:888-891.

1. A method for inducing an immune response to an antigen in a subject, the method comprising administering to the subject DNA encoding the antigen, and subsequently orally administering to the subject a composition comprising transgenic material, wherein the transgenic material comprises a DNA molecule encoding the antigen such that the antigen is expressed in the transgenic material.

2. A method as claimed in claim 1 in which the composition further comprises a mucosal adjuvant.

3. A method as claimed in claim 2 in which the mucosal adjuvant is cholera toxin β-subunits.

4. A method as claimed in any one of claims 1 to 3 in which the antigen is expressed in the transgenic material as a fusion protein.

5. A method as claimed in claim 4 in which the fusion protein comprises the antigen C-terminally fused to the amino acid sequence SEKDEL.

6. A method as claimed in any one of claims 1 to 5 in which the transgenic material is a transgenic plant.

7. A method as claimed in claim 6 in which the transgenic plant is a fruit or vegetable.

8. A method as claimed in claim 6 in which the transgenic plant is selected from the group consisting of; tobacco, lettuce, rice and bananas.

9. A method as claimed in any one of claims 1 to 8 in which the antigen is selected from the group consisting of viral antigens, parasitic antigens and bacterial antigens.

10. A method as claimed in claim 9 in the which the antigen is from measles virus, the human immunodeficiency virus, or Plasmodium sp.

11. A method as claimed in claim 10 in which the antigen is selected from the group consisting of the measles virus H or F protein, or fragments thereof.

12. A method as claimed in claim 11 in which the antigen is the measles H protein.

13. A method as claimed in any one of claims 1 to 12 in which the DNA encoding the antigen is administered only once to the subject.

14. A method as claimed in any one of claims 1 to 12 in which the DNA encoding the antigen is administered to the subject on at least two occasions.

15. A method as claimed in any one of claims 1 to 14 in which the composition comprising transgenic material is orally administered only once to the subject.

16. A method as claimed in any one of claims 1 to 14 in which the composition comprising transgenic material is orally administered to the subject on at least two occasions.

17. A transgenic plant, the plant having been transformed with a DNA molecule, the DNA molecule comprising a sequence encoding a measles virus antigen such that the plant expresses the measles virus antigen.

18. A transgenic plant as claimed in claim 17 in the DNA molecule encodes a fusion protein.

19. A transgenic plant as claimed in claim 18 in which the fusion protein comprises the measles antigen C-terminally fused to the amino acid sequence SEKDEL.

20. A transgenic plant as claimed in any one of claims 17 to 19 in which the measles antigen is the measles H protein.


SEQUENCE LISTING (7 sequences)

SEQ ID NO: 1 - protein, 6 aa, Artificial Sequence
  Description of Artificial Sequence ER retention signal
SEKDEL

SEQ ID NO: 2 - DNA, 30 bp, Artificial Sequence
  Description of Artificial Sequence oligonucleotide
tcgatctctgagaaagatgagctatgaggg

SEQ ID NO: 3 - DNA, 30 bp, Artificial Sequence
  Description of Artificial Sequence oligonucleotide
gatcccctcatagctcatctttctcagaga

SEQ ID NO: 4 - protein, 4 aa, Measles virus
TNRR

SEQ ID NO: 5 - protein, 10 aa, Artificial Sequence
  Description of Artificial Sequence C-terminal end of measles H protein fused with ER retention signal
TNLQSEKDEL

SEQ ID NO: 6 - DNA, 23 bp, Artificial Sequence
  Description of Artificial Sequence PCR primer
gcgccatgggatttgttctcttt

SEQ ID NO: 7 - DNA, 34 bp, Artificial Sequence
  Description of Artificial Sequence PCR primer
tatccatgggcccggcacggcaagagtgggatat